(19) United States
(12) Patent Application Publication
(10) Pub. No.: US 2008/0025889 A1
(43) Pub. Date: Jan. 31, 2008

(54) Steam-hose with steam-trap

(76) Inventor: Dwayne Brent Cole, Camden, TN (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 11/389,925

(22) Filed: Mar. 27, 2006

(51) Int. Cl. A61L 2/00

(52) U.S. Cl. 422/292; 122/15.1

(57) ABSTRACT

The embodiments of the present invention relate to a steam-hose device wherein the condensate accumulating in the steam-hose is in-line-discharged through a steam-trap. Embodiments of the present invention also relate to a process for in-line-discharging condensate accumulating in the steam-hose, through a steam-trap. Embodiments of the present invention further relate to a process for heating objects by spraying of steam or steam with another gas.

### FILED OF INVENTION

The embodiments of the present invention relate to a steam-hose device wherein the condensate accumulating in the steam-hose is in-line-discharged through a steam-trap. Embodiments of the present invention also relate to a process for in-line-discharging condensate accumulating in the steam-hose, through a steam-trap. Embodiments of the present invention further relate to a process for heating objects by spraying of steam or steam with another gas.

### BACKGROUND

In industrial settings and otherwise, steam is often used to heat equipment. During winter months and/or in colder environments, steam can condense in the steam-hose used for spraying steam. The accumulated condensate can even freeze to ice in the steam hose. As more water accumulates and freezes, the hose is blocked and can rupture due to the steam pressure and/or the expansion of water while freezing. In addition to replacement cost of the ruptured steam-hose and the down-time as a result of the replacement, a rupture in the hose can create unsafe operating conditions for the steam-hose operator because the steam is under pressure and at high temperature.

In order to prevent such condensate from blocking the steam-hose and causing the rupture, a small amount of steam must be constantly vented so that the steam-hose and the attached steam-lance remain hot. This reduces the condensation of water and also expels any condensed water. However, this results in a substantial energy loss when the steam-hose is not being used.

On the other hand, the steam-hose can be shut-down to avoid energy loss. However, if, when the steam is turned back on, the condensate can evaporate suddenly, also causing a rupture in the steam-hose. This situation can also create an unsafe operating condition for the operator because the steam is under pressure and at high temperature.

U.S. Pat. No. 4,674,977 describes a method for overcoming this problem. A drum-like container stores the steam-hose in an internal chamber that is heated by steam passing through a heating coil. Separate valves control the steam supplied to the heating coil and the steam-hose. Lengths of the hose can remain coiled in the drum while the free end of the steam-hose supplies steam to desired locations. However, clearly, it is a cumbersome method to overcome the problem. Furthermore, when the steam-hose is moved, the drum also needs to be moved. Also, energy is wasted in heating the chamber on a continuous basis.

The present invention provides a steam-hose device that addresses the above problems. Particularly, the device provides a method that allows a steam-hose to remain hot during the winter months and/or in cold environments. Particularly, the invention provides a steam-trap on the steam-lance at the free end of the steam-hose. The steam is routed through the steam-trap when the steam-lance valve is closed. Condensate water is removed from the system through the steam-trap and discharged in-line.

Consequently, hot steam remains in the steam hose, keeping the hose ready for use at all times. As discussed previously, without a steam-trap the steam-lance must vent steam to remain hot, resulting in wasted steam loss.

### SUMMARY OF INVENTION

This invention relates to a steam-hose device, comprising:
(A) a steam-hose, having a first end and a second end;
(B) a Y-shaped coupling, having a first outlet, a second outlet, and a third outlet, wherein said third outlet of said Y-shaped coupling is detachably connected to said first end of said steam-hose by means for connecting said third outlet of said Y-shaped coupling to said first end of said steam-hose, and wherein, optionally, said Y-shaped coupling has attached to it, a back handle;
(C) a valve, having a first end and a second end, wherein said first end of said valve is detachably connected to said first outlet of said Y-shaped coupling by means for connecting said first end of said valve to said first outlet of said Y-shaped coupling;
(D) a return tube, having a first end and a second end, wherein said first end of said return tube is detachably connected to said second outlet of said Y-shaped coupling by means for connecting said first end of said return tube to said second outlet of said Y-shaped coupling;
(E) a steam-trap, having a first end and a second end, wherein said first end of said steam-trap is detachably connected to said second end of said return tube by means for connecting said first end of said steam-trap to said second end of said return tube;
(F) an angular pipe, having a first end and a second end, wherein said first end of said angular pipe is detachably connected to said second end of said steam-trap by means for connecting said first end of said angular pipe to said second end of said steam-trap; and
(G) a steam-lance having a first end and a second end,
   wherein said first end of said steam-lance is detachably connected to said second end of said valve by means for connecting said first end of said steam-lance to said second end of said valve;
   wherein said second end of said angular pipe is optionally detachably connected to said steam-lance by means for connecting said second end of said angular pipe to said steam-lance;
   wherein said second end of said steam-lance is open to environment and wherein said second end of said steam-lance is tapered;
   wherein said steam-lance is optionally covered with an insulating sleeve; and
   wherein optionally, said Y-shaped coupling has attached to it, a front handle.
      This invention further relates to a process for using a steam-hose device, wherein said steam-hose device comprises:
(A) a steam-hose, having a first end and a second end;
(B) a Y-shaped coupling, having a first outlet, a second outlet, and a third outlet, wherein said third outlet of said Y-shaped coupling is detachably connected to said first end of said steam-hose by means for connecting said third outlet of said Y-shaped coupling to said first end of said steam-hose and wherein, optionally, said Y-shaped coupling has attached to it, a back handle;
(C) a valve, having a first end and a second end, wherein said first end of said valve is detachably connected to said first outlet of said Y-shaped coupling by means for connecting said first end of said valve to said first outlet of said Y-shaped coupling;
(D) a return tube, having a first end and a second end, wherein said first end of said return tube is detachably connected to said second outlet of said Y-shaped coupling by means for connecting said first end of said return tube to said second outlet of said Y-shaped coupling;
(E) a steam-trap, having a first end and a second end, wherein said first end of said steam-trap is detachably connected to said second end of said return tube by means for connecting said first end of said steam-trap to said second end of said return tube;
(F) an angular pipe, having a first end and a second end, wherein said first end of said angular pipe is detachably connected to said second end of said steam-trap by means for connecting said first end of said angular pipe to said second end of said steam-trap;
(G) a steam-lance having a first end and a second end,
   wherein said first end of said steam-lance is detachably connected to said second end of said valve by means for connecting said first end of said steam-lance to said second end of said valve;
   wherein said second end of said angular pipe is optionally detachably connected to said steam-lance by means for connecting said second end of said angular pipe to said steam-lance;
   wherein said second end of said steam-lance is open to environment and wherein said second end of said steam-lance is tapered;
   wherein said steam-lance is optionally covered with an insulating sleeve; and
   wherein optionally, said steam-lance has attached to it, a front handle.
      This invention also relates to a process for heating an object, comprising the steps of:
      (I) supplying steam or a combination of steam and another gas to a steam-hose device, wherein said steam hose device comprises:
(A) a steam-hose, having a first end and a second end;
(B) a Y-shaped coupling, having a first outlet, a second outlet, and a third outlet, wherein said third outlet of said Y-shaped coupling is detachably connected to said first end of said steam-hose by means for connecting said third outlet of said Y-shaped coupling to said first end of said steam-hose and wherein, optionally, said Y-shaped coupling has attached to it, a back handle;
(C) a valve in a closed position, having a first end and a second end, wherein said first end of said valve is detachably connected to said first outlet of said Y-shaped coupling by means for connecting said first end of said valve to said first outlet of said Y-shaped coupling;
(D) a return tube, having a first end and a second end, wherein said first end of said return tube is detachably connected to said second outlet of said Y-shaped coupling by means for connecting said first end of said return tube to said second outlet of said Y-shaped coupling;
(E) a steam-trap, having a first end and a second end, wherein said first end of said steam-trap is detachably connected to said second end of said return tube by means for connecting said first end of said steam-trap to said second end of said return tube;
(F) an angular pipe, having a first end and a second end, wherein said first end of said angular pipe is detachably connected to said second end of said steam-trap by means for connecting said first end of said angular pipe to said second end of said steam-trap;
(G) a steam-lance having a first end and a second end,
   wherein said first end of said steam-lance is detachably connected to said second end of said valve by means for connecting said first end of said steam-lance to said second end of said valve;
   wherein said second end of said angular pipe is optionally detachably connected to said steam-lance by means for connecting said second end of said angular pipe to said steam-lance;
   wherein said second end of said steam-lance has an opening to the environment;
   wherein said steam-lance is optionally covered with an insulating sleeve; and
   wherein optionally, said steam-lance has attached to it, a front handle.
   wherein said another gas is selected from the group consisting of air, nitrogen, helium, and combinations thereof;
      (II) opening said steam-trap and discharging condensed steam and/or water through said opening at said second end of said steam-lance;
      (III) closing said steam-trap;
      (IV) opening said valve;
      (V) leading said steam or said steam and said another gas into said steam-lance and out from said opening at said second end of said steam-lance;
      (VI) spraying said object with said steam or said steam and said another gas; and
      (VII) closing said valve.
         This invention also relates to a process for removing condensate from a steam-hose device, comprising the steps of:
      (I) providing a steam-hose, having a first end and a second end;
      (II) providing a Y-shaped coupling, having a first outlet, a second outlet, a third outlet; and, optionally providing said Y-shaped coupling with a back handle;
      (III) detachably connecting said third outlet of said Y-shaped coupling to said first end of said steam-hose by means for connecting said third outlet of said Y-shaped coupling to said first end of said steam-hose;
      (IV) providing a valve in a closed position, having a first end and a second end;
      (V) detachably connecting said first end of said valve to said first outlet of said Y-shaped coupling by means for connecting said first end of said valve to said first outlet of said Y-shaped coupling;
      (VI) providing a return tube, having a first end and a second end;
      (VII) detachably connecting said first end of said return tube to said second outlet of said Y-shaped coupling by means for connecting said first end of said return tube to said second outlet of said Y-shaped coupling;
      (VIII) providing a steam-trap, having a first end and a second end;
      (IX) detachably connecting said first end of said steam-trap to said second end of said return tube by means for connecting said first end of said steam-trap to said second end of said return tube;
      (X) providing an angular pipe, having a first end and a second end;
      (XI) detachably connecting said first end of said angular pipe to said second end of said steam-trap by means for connecting said first end of said angular pipe to said second end of said steam-trap;
      (XII) providing a steam-lance having a first end and a second end, wherein said second end having an opening to the environment, and optionally covering said steam-lance with an insulating sleeve and wherein optionally, said steam-lance has attached to it, a front handle;
      (XIII) detachably connecting said first end of said steam-lance to said second end of said valve by means for connecting said first end of said steam-lance to said second end of said valve;
      (XIV) optionally-detachably connecting said second end of said angular pipe to said steam-lance by means for connecting said second end of said angular pipe to said steam-lance;
      (XV) supplying steam or a combination of steam and another gas to a steam-hose device, wherein said another gas is selected from the group consisting of air, nitrogen, helium, and combinations thereof;
      (XVI) opening said steam-trap and discharging condensed steam and/or water through said opening at said second end of said steam-lance; and
      (XVII) closing said steam-trap.

### BRIEF DESCRIPTION OF DRAWINGS

1. FIG. 1 depicts a the steam-hose device.

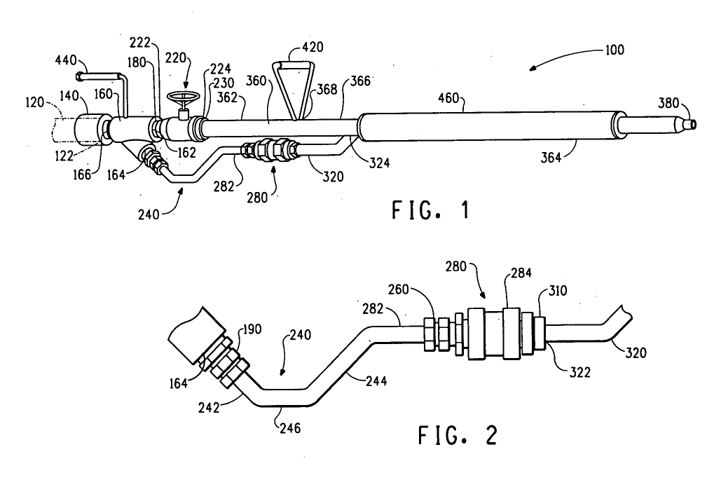

2. FIG. 2 depicts an exemplary the return tube.

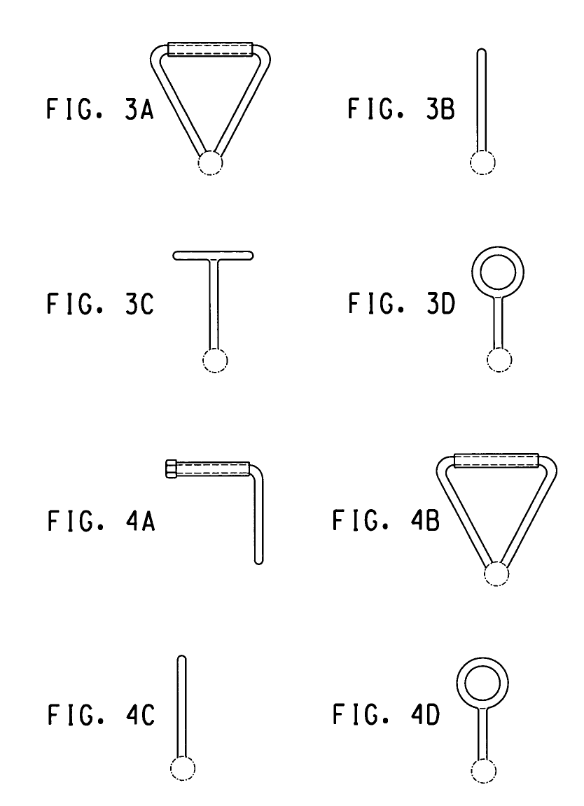

3. FIG. 3 depicts examples of the front handle.

4. FIG. 4 depicts examples of the back handle.

### DETAILED DESCRIPTION OF THE INVENTION

By “condensate” is meant the water and/or ice that has condensed from the steam.

By “in-line-discharge” is meant that the steam or water are removed from the steam-hose device through the free-end of the steam-hose, which is the tapering end of the steam-lance from which steam is sprayed.

By “steam-hose device” is meant the entire steam-lance assembly, including the steam hose, the Y-shaped coupling, the valve, the steam-trap, the steam-lance, etc.

The embodiments of the present invention relate to a steam-hose device wherein the condensate accumulating in the steam-hose is in-line-discharged through a steam-trap. Embodiments of the present invention also relate to a process for in-line-discharging of condensate accumulating in the steam-hose, through a steam-trap. Embodiments of the present invention further relate to a process for heating objects by spraying of steam or steam and another gas.

During winter months or in colder environments, in a steam-hose not in use, steam can condense to water and even freeze to ice. This can result into the rupture of the steam-hose and also create an obstruction to using the steam at desired time. Furthermore, when the steam is turned back on, accumulated water or ice can evaporate suddenly causing unsafe operating conditions for the operator.

As a result, generally, steam is constantly bled through the steam-lance to keep the steam-hose and the attached parts sufficiently warm to preclude freezing and/or condensation. Clearly, this results in energy loss in form of hot steam and also inefficient operation.

The present invention relates to incorporating a steam-trap parallel to the steam-lance in such a manner that the condensate is discharged in-line. The embodiments of the invention are described in detail below.

FIG. 1 describes the general assembly of the steam-hose device (100). The steam-hose (120) is available to draw steam from a wall-outlet (not shown) or any other source of steam (not shown). The steam-hose (120) is made from standard materials such as plastic or cloth, optionally reinforced with metal. Such steam-hoses are known to one skilled in the art. Generally, the steam-hose (120) is flexible, so that it can be to moved different locations for application of steam.

The steam-hose (120) has a first end (122) and a second end (not shown). The first end (122) of the steam-hose (120) is connected to a Y-shaped coupling (160). The Y-shaped coupling (160) has three outlets, first outlet (162), second outlet (164) and the third outlet (166). The first end (122) of the steam-hose (120) is detachably connected by connecting means (140) with the third outlet (166) of the Y-shaped coupling (160), as shown in FIG. 1. Generally, any standard connecting means known to a person of ordinary skill in the pertinent art can be used for the particular connecting means (140). In one embodiment, the connecting means (140) could be a simple arrangement wherein the third outlet (166) of the Y-shaped coupling (160) is simply screwed onto the steam-hose (120). Clearly, other connecting means (140) can include, for example, a valve and coupling with a hose-clamp. In a simplest form, the connecting means (140) could also be gluing the Y-shaped coupling (140) to the steam-hose (120), or affixing the Y-shaped coupling (140) on to the steam-hose (120) without any screw action but with an ordinary clamp. In other words, the inventor clearly envisages as part of his invention the various embodiments that can be used as connecting means (140) for the steam-hose (120) and the Y-shaped coupling (160).

The first outlet (162) of the Y-shaped coupling (160) is detachably connected to a valve (220) through its first end (222) by connecting means (180). The valve (220) can be used to control the flow of steam from the steam-hose to the object or equipment on which it is sprayed on. The valve (220) can be fully closed or fully open. The valve (220) can be threaded into the Y-shaped coupling (160). Generally, any standard connecting means known to a person of ordinary skill in the pertinent art can be used for the particular connecting means (180). Clearly, other connecting means (180) can include, for example, a valve and coupling with a hose-clamp. In a simplest form, the connecting means (180) could also be gluing the Y-shaped coupling (160) to the first end (222) of the valve (220), or affixing the Y-shaped coupling (160) on to the first end (222) of the valve (220) without any screw action but with an ordinary clamp. In other words, the inventor clearly envisages as part of his invention the various embodiments that can be used as connecting means (180) for the valve (220) and the Y-shaped coupling (160).

The second outlet (164) of the Y-shaped coupling (160) is detachably connected to a return tube (240) as shown in FIG. 2. The return tube (240) is generally a pipe with a first end (242), a middle portion (246) and a second end (244). The return tube (240) is connected to the second outlet (164) of the Y-shaped coupling (160) through its first end (242) by connecting means (190). Generally, any standard connecting means known to a person of ordinary skill in the pertinent art can be used for the particular connecting means (190). Clearly, other connecting means (190) can include, for example, a valve and coupling with a hose-clamp. In a simplest form, the connecting means (190) could also be gluing the Y-shaped coupling (160) to the first end (242) of the return tube (240), or affixing the Y-shaped coupling (160) on to the first end (242) of the return tube (240) without any screw action but with an ordinary clamp. In other words, the inventor clearly envisages as part of his invention the various embodiments that can be used as connecting means (190) for the valve (240) and the Y-shaped coupling (160). In one embodiment, the connecting means (190) is a compression fitting in conjunction with a hex-head bushing.

In one embodiment, as shown in FIG. 2, the return tube is S-shaped and comprises the first end (242) that is at about 45° angle to the middle portion (246) of the return tube (240). The middle portion (246) is at an angle of about 45° angle to the second end (244) of the return tube (240). In one embodiment, the return tube (240) is made of metal. The return tube (240) can also be made of plastic. The return tube (240) need not be in an S-shape. The return tube (240) can be flexible, rigid, or semi-rigid.

As depicted in FIGS. 1 and 2, the return tube (240), at its second end (244), is detachably connected to a first end (282) of a steam-trap (280) through connecting means (260). The steam-trap (280) is a standard steam-trap known to a person of ordinary skill in the pertinent art. The steam-trap (280) can be a temperature sensitive steam-trap or a pressure sensitive steam-trap. Thus the steam-trap (280) will automatically open at a particular temperature to discharge the condensate or it will automatically open at a particular pressure to discharge the condensate. The steam-trap (280) can be calibrated to open at specified temperature or pressure. The steam-trap (280) can also be calibrated to open and close automatically at a specified time interval to discharge the condensate (when the steam-hose device is not in operation). By closing the steam-trap (280) is meant that the steam-trap (280) is bypassed by the steam, and in that position, the steam flows through directly from the Y-shaped coupling (160) into the steam lance (360). Steam-traps are known to a person of ordinary skill in the pertinent art. A steam-trap could be obtained from Spirax Sarco, Ltd., Cheltenham, United Kingdom.

Generally, any standard connecting means known to a person of ordinary skill in the pertinent art can be used for the particular connecting means (260). Clearly, other connecting means (260) can include, for example, a valve and coupling with a hose-clamp. In a simplest form, the connecting means (260) could also be gluing the steam-trap (280) to the second end (244) of the return tube (240), or affixing the steam-trap (280) on to the second end (244) of the return tube (240) without any screw action but with an ordinary clamp. The steam-trap (280) can also be simply screwed onto the threads at the second end (244) of the return tube (240). In other words, the inventor clearly envisages as part of his invention the various embodiments that can be used as connecting means (260) for the steam-trap (280) and the second end (244) of the return tube (240). In one embodiment, the connecting means (260) is a compression fitting in conjunction with a hex-head bushing.

Generally, a steam-trap discharges condensate while not permitting the escape of live steam. The steam-trap purges the condensate out of the system allowing steam to reach its destination in as dry a state/condition as possible. The quantity of condensate a steam-trap has to deal with may vary considerably. It may have to discharge condensate at steam temperature (i.e. as soon as it forms in the steam space) or it may be required to discharge below steam temperature. The pressures at which steam-traps can operate may be anywhere from vacuum to well over a hundred bar.

As shown in FIGS. 1 and 2, the second end (284) of the steam-trap (280) is detachably connected to first end (322) of an angular pipe (320) through connecting means (310). Generally, any standard connecting means known to a person of ordinary skill in the pertinent art can be used for the particular connecting means (310). Clearly, other connecting means (310) can include, for example, a valve and coupling with a hose-clamp. In a simple form, the connecting means (310) could also be gluing the steam-trap (280) to the first end (322) of the angular pipe (320), or affixing the steam-trap (280) on to the first end (322) of the return tube (320) without any screw action but with an ordinary clamp. The steam-trap (280) can also be simply screwed onto the threads at the first end (322) of the return tube (320). In other words, the inventor clearly envisages as part of his invention the various embodiments that can be used as connecting means (310) for the steam-trap (280) and the angular pipe (320). In one embodiment, the connecting means (310) is a compression fitting in conjunction with a hex-head bushing.

The angular pipe (320) is generally made of a metal. However, it can be made of plastic or rubber.

The second end (324) of the angular pipe (320) is welded to the steam-lance (360). It can also be detachably connected to the steam-lance (360). The steam-lance (360) comprises a first end (362), a second end (364) and a middle portion (366). The second end (324) of the angular pipe (320) is connected to the steam-lance (360) at its middle portion (366). The steam-lance (360) is generally a straight pipe, generally bigger in diameter than either the return tube (240) or the angular pipe (320).

The first end (362) of the steam-lance (360) is detachably connected to the second end (224) of the valve (220) through connecting means (230). Generally, any standard connecting means known to a person of ordinary skill in the pertinent art can be used for the particular connecting means (230). Clearly, other connecting means (230) can include, for example, a valve and coupling with a hose-clamp. In a simplest form, the connecting means (230) could also be gluing the valve (220) to the first end (362) of the angular pipe (360), or affixing the valve (220) on to the first end (362) of the steam-lance (360) without any screw action but with an ordinary clamp. The valve (220) can also be simply screwed onto the threads at the first end (362) of the steam-lance (360). In other words, the inventor clearly envisages as part of his invention the various embodiments that can be used as connecting means (230) for the valve (220) and the steam-lance (360). In one embodiment, the connecting means (230) is a compression fitting in conjunction with a hex-head bushing.

The second end (364) of the steam-lance is tapered at the tip (380). The second end (364) of the steam-lance is the free-end at the tip (380) from which the steam and/or the condensate are discharged. From about the middle portion (366) to about its second end (364), the steam-lance (360) may be optionally covered with an insulating sleeve (460) that may aid the operator while applying steam. It may also reduce heat loss by providing insulation.

In one embodiment of the invention, about in between the middle portion (366) and the first end (362) of the steam-lance (360), a front handle (420) is attached, generally by welding (368), to form one piece with the steam-lance (360). The front handle (420) can be in the shape of an inverse triangle when the steam-hose device (100) is in operation. The front handle (420) can also be in shape of a “T”. The front handle (420) can also be in shape of a rod, or the shape of a rod with a closed loop at the top. Exemplary front handles (420) are shown in FIG. 3. FIG. 3A through 3D describe exemplary handles that can be used in the present invention. It should be noted that such front handles are only exemplary of what is within the scope of the invention. For example, such front handles (420) could have a rubber, plastic, or a metal sleeve that would help with the grip on the handle and/or insulation.

Optionally, a back handle (440) can also be attached to the Y-shaped coupling (160). The back handle (440) can be welded to the Y-shaped coupling (160) to form one piece with the Y-shaped coupling (160). The back handle (440) can be “L-shaped”, triangle shaped, rod-shaped or in shape of a rod with a closed loop at the end of the handle. Exemplary shapes are given in FIG. 4A through 4D.

Such steam-hose device (100) can be used to heat an object by spraying steam on to the said object. Such objects could be an industrial equipment. This invention also relates to the process of heating such objects.

The process for heating an object, comprises supplying steam or a combination of steam and another gas to the steam-hose device, wherein said another gas is selected from the group consisting of air, nitrogen, helium, and combinations thereof. When the steam-hose device (100) is not in use, the valve (220) is in a closed position and no steam emanates from the second end (364) of the steam-lance (360). The return tube (240) leading to the steam-trap (280) always remains open. In this situation, i.e., when the steam-hose device (100) is not in use, the condensate accumulating in the Y-shaped coupling (160) and the return tube (240) is discharged in-line through the second-end (364) of the steam-lance (360) because the steam-trap (280) opens up periodically for this action (the valve (220) is in a closed position and therefore no steam passes through it and into the steam-lance (360). As discussed previously, the steam-trap can open up as a result of temperature-change, pressure-change, or periodically. Such opening up of the steam-trap (280) can be programmed by the user, usually, a person of ordinary skill in the pertinent art. Once the condensate is discharged through the second end (364) of the steam-lance (360) at the tip of the steam-lance (380), the steam-trap (280) closes. The valve (220) can be opened to lead the steam through the valve (220) and through the second end (364) of the steam-lance (360) to impinge on to a desirable object without any problems, or unsafe conditions, discussed previously. The possibility of rupture of the steam-hose (120) is also greatly reduced as a result of the discharge of the condensate. The front handle (420) and/or the back handle (440) can be used to direct the impingement of steam said steam and said another gas on to the desired object. Once the application of steam is completed, the valve (220) is closed and the steam-hose device (100) is now not in use. This cycle is repeated.

This invention also relates to using such steam-hose device for heating equipment, industrial equipment, in clothes laundering to iron clothes, in spraying steam in household applications, and the like. These are exemplary embodiments of use of such steam-hose device. In general, the steam-hose device can be used in such situations where there is a need to spray steam.

What is claimed is:

1. A steam-hose device, comprising:
(A) a steam-hose, having a first end and a second end;
(B) a Y-shaped coupling, having a first outlet, a second outlet, and a third outlet, wherein said third outlet of said Y-shaped coupling is detachably connected to said first end of said steam-hose by means for connecting said third outlet of said Y-shaped coupling to said first end of said steam-hose, and wherein, optionally, said Y-shaped coupling has attached to it, a back handle;
(C) a valve, having a first end and a second end, wherein said first end of said valve is detachably connected to said first outlet of said Y-shaped coupling by means for connecting said first end of said valve to said first outlet of said Y-shaped coupling;
(D) a return tube, having a first end and a second end, wherein said first end of said return tube is detachably connected to said second outlet of said Y-shaped coupling by means for connecting said first end of said return tube to said second outlet of said Y-shaped coupling;
(E) a steam-trap, having a first end and a second end, wherein said first end of said steam-trap is detachably connected to said second end of said return tube by means for connecting said first end of said steam-trap to said second end of said return tube;
(F) an angular pipe, having a first end and a second end, wherein said first end of said angular pipe is detachably connected to said second end of said steam-trap by means for connecting said first end of said angular pipe to said second end of said steam-trap; and
(G) a steam-lance having a first end and a second end,
wherein said first end of said steam-lance is detachably connected to said second end of said valve by means for connecting said first end of said steam-lance to said second end of said valve;
wherein said second end of said angular pipe is optionally detachably connected to said steam-lance by means for connecting said second end of said angular pipe to said steam-lance;
wherein said second end of said steam-lance is open to environment and wherein said second end of said steam-lance is tapered;
wherein said steam-lance is optionally covered with an insulating sleeve; and
wherein optionally, said Y-shaped coupling has attached to it, a front handle.

2. A process for using a steam-hose device, wherein said steam-hose device comprises:
(A) a steam-hose, having a first end and a second end;
(B) a Y-shaped coupling, having a first outlet, a second outlet, and a third outlet, wherein said third outlet of said Y-shaped coupling is detachably connected to said first end of said steam-hose by means for connecting said third outlet of said Y-shaped coupling to said first end of said steam-hose and wherein, optionally, said Y-shaped coupling has attached to it, a back handle;
(C) a valve, having a first end and a second end, wherein said first end of said valve is detachably connected to said first outlet of said Y-shaped coupling by means for connecting said first end of said valve to said first outlet of said Y-shaped coupling;
(D) a return tube, having a first end and a second end, wherein said first end of said return tube is detachably connected to said second outlet of said Y-shaped coupling by means for connecting said first end of said return tube to said second outlet of said Y-shaped coupling;
(E) a steam-trap, having a first end and a second end, wherein said first end of said steam-trap is detachably connected to said second end of said return tube by means for connecting said first end of said steam-trap to said second end of said return tube;
(F) an angular pipe, having a first end and a second end, wherein said first end of said angular pipe is detachably connected to said second end of said steam-trap by means for connecting said first end of said angular pipe to said second end of said steam-trap;
(G) a steam-lance having a first end and a second end,
wherein said first end of said steam-lance is detachably connected to said second end of said valve by means for connecting said first end of said steam-lance to said second end of said valve;
wherein said second end of said angular pipe is optionally detachably connected to said steam-lance by means for connecting said second end of said angular pipe to said steam-lance;
wherein said second end of said steam-lance is open to environment and wherein said second end of said steam-lance is tapered;
wherein said steam-lance is optionally covered with an insulating sleeve; and
wherein optionally, said steam-lance has attached to it, a front handle.

3. A process for heating an object, comprising the steps of:
(I) supplying steam or a combination of steam and another gas to a steam-hose device, wherein said steam hose device comprises:
(A) a steam-hose, having a first end and a second end;
(B) a Y-shaped coupling, having a first outlet, a second outlet, and a third outlet, wherein said third outlet of said Y-shaped coupling is detachably connected to said first end of said steam-hose by means for connecting said third outlet of said Y-shaped coupling to said first end of said steam-hose and wherein, optionally, said Y-shaped coupling has attached to it, a back handle;
(C) a valve in a closed position, having a first end and a second end, wherein said first end of said valve is detachably connected to said first outlet of said Y-shaped coupling by means for connecting said first end of said valve to said first outlet of said Y-shaped coupling;
(D) a return tube, having a first end and a second end, wherein said first end of said return tube is detachably connected to said second outlet of said Y-shaped coupling by means for connecting said first end of said return tube to said second outlet of said Y-shaped coupling;
(E) a steam-trap, having a first end and a second end, wherein said first end of said steam-trap is detachably connected to said second end of said return tube by means for connecting said first end of said steam-trap to said second end of said return tube;
(F) an angular pipe, having a first end and a second end, wherein said first end of said angular pipe is detachably connected to said second end of said steam-trap by means for connecting said first end of said angular pipe to said second end of said steam-trap;
(G) a steam-lance having a first end and a second end,
wherein said first end of said steam-lance is detachably connected to said second end of said valve by means for connecting said first end of said steam-lance to said second end of said valve;
wherein said second end of said angular pipe is optionally detachably connected to said steam-lance by means for connecting said second end of said angular pipe to said steam-lance;
wherein said second end of said steam-lance has an opening to the environment;
wherein said steam-lance is optionally covered with an insulating sleeve; and
wherein optionally, said steam-lance has attached to it, a front handle.
wherein said another gas is selected from the group consisting of air, nitrogen, helium, and combinations thereof;
(II) opening said steam-trap and discharging condensed steam and/or water through said opening at said second end of said steam-lance;
(III) closing said steam-trap;
(IV) opening said valve;
(V) leading said steam or said steam and said another gas into said steam-lance and out from said opening at said second end of said steam-lance;
(VI) spraying said object with said steam or said steam and said another gas; and
(VII) closing said valve.

4. A process for removing condensate from a steam-hose device, comprising the steps of:
(I) providing a steam-hose, having a first end and a second end;
(II) providing a Y-shaped coupling, having a first outlet, a second outlet, a third outlet; and, optionally providing said Y-shaped coupling with a back handle;
(III) detachably connecting said third outlet of said Y-shaped coupling to said first end of said steam-hose by means for connecting said third outlet of said Y-shaped coupling to said first end of said steam-hose;
(IV) providing a valve in a closed position, having a first end and a second end;
(V) detachably connecting said first end of said valve to said first outlet of said Y-shaped coupling by means for connecting said first end of said valve to said first outlet of said Y-shaped coupling;
(VI) providing a return tube, having a first end and a second end;
(VII) detachably connecting said first end of said return tube to said second outlet of said Y-shaped coupling by means for connecting said first end of said return tube to said second outlet of said Y-shaped coupling;
(VIII) providing a steam-trap, having a first end and a second end;
(IX) detachably connecting said first end of said steam-trap to said second end of said return tube by means for connecting said first end of said steam-trap to said second end of said return tube;
(X) providing an angular pipe, having a first end and a second end;
(XI) detachably connecting said first end of said angular pipe to said second end of said steam-trap by means for connecting said first end of said angular pipe to said second end of said steam-trap;
(XII) providing a steam-lance having a first end and a second end, wherein said second end having an opening to the environment, and optionally covering said steam-lance with an insulating sleeve and wherein optionally, said steam-lance has attached to it, a front handle;
(XIII) detachably connecting said first end of said steam-lance to said second end of said valve by means for connecting said first end of said steam-lance to said second end of said valve;
(XIV) optionally-detachably connecting said second end of said angular pipe to said steam-lance by means for connecting said second end of said angular pipe to said steam-lance;
(XV) supplying steam or a combination of steam and another gas to a steam-hose device, wherein said another gas is selected from the group consisting of air, nitrogen, helium, and combinations thereof;
(XVI) opening said steam-trap and discharging condensed steam and/or water through said opening at said second end of said steam-lance; and
(XVII) closing said steam-trap.
